(19) United States
(12) Patent Application Publication
(10) Pub. No.: US 2024/0425576 A1
(43) Pub. Date: Dec. 26, 2024

(54) METHOD FOR TREATING A PERIOHERAL ARTERIAL DISEASE

(71) Applicant: NATIONAL YANG MING CHIAO TUNG UNIVERSITY, Taipei (TW)

(72) Inventors: Ting-Ting CHANG, Taipei City (TW); Jaw-Wen CHEN, Taipei City (TW)

(73) Assignee: NATIONAL YANG MING CHIAO TUNG UNIVERSITY, Taipei (TW)

(21) Appl. No.: 18/214,178

(22) Filed: Jun. 26, 2023

(51) Int. Cl. C07K 16/24; A61K 9/00; A61P 9/10

(52) U.S. Cl. CPC C07K 16/24; A61K 9/0019; A61P 9/10

(57) ABSTRACT

Provided is a method for preventing or treating a peripheral arterial disease (PAD) in a subject in need thereof, including administering an effective amount of a chemokine C-C motif ligand 7 (CCL7) antagonist to the subject to inhibit CCL7 activity.

### BACKGROUND

### 1. Technical Field

The present disclosure relates to a method for improving a peripheral arterial disease (PAD), and particularly to methods for preventing or treating PAD in a subject suffering from diabetes.

### 2. Description of Related Art

Peripheral arterial disease (PAD), also known as peripheral artery disease, refers to partial or complete occlusion of the peripheral vessels of the upper and lower limbs. The prevalence of PAD is expected to continue to increase in the foreseeable future owing to the rise in the occurrence of its major risk factors. Non-healing ulcers, limb amputation, and physical disability are some significant complications.

Diabetes mellitus (DM) is a chronic metabolic disease characterized by elevated blood glucose levels. Diabetic vascular complications are associated with endothelial dysfunction and impaired angiogenesis. Diabetes mellitus (DM) remains a significant risk factor for PAD, and DM patients have an increased prevalence of PAD compared to the general population. The clinical presentation in people with DM also differs slightly from that of the general population. In addition, PAD in DM can lead to diabetic foot ulcers (DFUs), which trigger hyperglycemic emergencies and result in increased hospitalizations, decreased quality of life, and mortality. Despite the epidemiologic and clinical importance of PAD, it remains largely underdiagnosed and therefore undertreated, possibly because it is largely asymptomatic.

The chemokines (or chemotactic cytokines) are a large family of small, secreted proteins that signal through cell surface G protein-coupled heptahelical chemokine receptors. Previous studies have shown that chemokines play a critical role in inflammation and immunity. Some inflammatory chemokines have been implicated as potential contributors to and therapeutic targets for cardiovascular disease (CVD) and diabetes. Chemokine C-C motif ligand 7 (CCL7), also known as monocyte-specific chemokine (MCP)-3, is a member of the C-C chemokine ligand family. In addition, CCL7 levels were upregulated in patients with type 2 diabetes mellitus (T2DM). However, the specific role of CCL7 in diabetic vascular disease remains unknown.

Hence, there is still an unmet need for improved prevention or treatment of PAD.

### SUMMARY

In view of the foregoing, the present disclosure provides a method for preventing or treating peripheral arterial disease (PAD) in a subject in need thereof, comprising administering an effective amount of a chemokine C-C motif ligand 7 (CCL7) antagonist to the subject to inhibit CCL7 activity.

In one embodiment of the present disclosure, the CCL7 antagonist is selected from a group consisting of a CCL7 neutralizing antibody, a CCL7 RNA interference (RNAi) agent, a C-C chemokine receptor type 1 antagonist, a C-C chemokine receptor type 2 antagonist, a C-C chemokine receptor type 3 antagonist, a C-C chemokine receptor type 5 antagonist, and a combination thereof.

In one embodiment of the present disclosure, the subject suffers from a disease inducing occlusion or potentiate stenosis of peripheral arteries. In another embodiment of the present disclosure, the disease inducing occlusion or potentiate stenosis of peripheral arteries can be any diseases or conditions that may induce acute or chronic artery occlusion.

In one embodiment of the present disclosure, the peripheral arterial disease is induced by at least one selected from the group consisting of diabetes, hypertension, radiation, toxins, atherosclerosis, thrombo-embolism, traumatic injury, vascular spasm, autoimmune disease, scleroderma, and vasculitis.

In one embodiment of the present disclosure, the diabetes is type 2 diabetes mellitus (T2DM).

In one embodiment of the present disclosure, the subject suffers from T2DM with vascular endothelial cell damages.

In one embodiment of the present disclosure, the subject is a human or an animal.

In one embodiment of the present disclosure, the effective amount of the CCL7 antagonist is from about 0.01 μg/kg to about 100 mg/kg. In another embodiment of the present disclosure, the effective amount of the CCL7 antagonist is from about 0.1 μg/kg to about 1 mg/kg.

In one embodiment of the present disclosure, the CCL7 antagonist is administered to the subject orally, intraperitoneally, intravenously, intradermally, intramuscularly, subcutaneously, intrapleurally, or transdermally.

In one embodiment of the present disclosure, the administering enhances angiogenesis in the subject. In another embodiment of the present disclosure, the administering protects endothelial cell functions in the subject. In yet another embodiment of the present disclosure, the administering improves a diabetic vascular disease.

In the present disclosure, by using the CCL7 antagonist, the method provided in the present disclosure may improve the functions of endothelial progenitor cells (EPCs) and human aortic endothelial cells (HAECs), so as to enhance angiogenesis. The method of using a CCL7 antagonist of the present discourse is useful in accelerating angiogenic process, and thus effective in treating PAD.

Other objectives, advantages and novel features of the invention will become more apparent from the following detailed description when taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following description of the embodiments, with reference made to the accompanying drawings.

FIGS. 1A to 1C are graphs illustrating that the CCL7 expression was increased and the angiogenic factor expressions were decreased in HAECs by the high glucose stimulation. The levels of CCL7 in the supernatants of HAECs (n=6; FIG. 1A) and CCL7 expression was up regulated in HAECs under the high glucose conditions (n=3; FIG. 1B). Western blotting and statistical analyses of p-eNOS, p-AKT, VEGF, and SDF-1 expressions in the high glucose induced HAECs (n=3; FIG. 1C). 1D represents the high glucose treatment for 1 day; 2D represents the high glucose treatment for 2 days; 3D represents the high glucose treatment for 3 days; M represents the mannitol treatment. *P<0.05, **P<0.01.

FIGS. 2A to 2D are graphs illustrating that the angiogenic factors and the endothelial functions were improved in HAECs under the high glucose conditions via inhibition of CCL7. Western blotting and statistical analyses of CCL7 expression after administration of CCL7 siRNA for 2 days in the high glucose-treated HAECs (n=3; FIG. 2A). The tube formation and migration abilities after administration of CCL7 siRNA for 2 days in the high glucose-treated HAECs (n=3; FIG. 2B, FIG. 2C). Western blotting and statistical analyses of p-eNOS, p-AKT, VEGF, and SDF-1 expressions after administration of CCL7 siRNA for 2 days in the high glucose-treated HAECs (n=3; FIG. 2D). M represents the mannitol treatment. *P<0.05, **P<0.01.

FIGS. 3A to 3D are graphs illustrating that the CCL7 level was enhanced, and the angiogenic factors were reduced in EPCs from T2DM patients. The level of CCL7 in plasma from healthy volunteers and T2DM patients (n=5; FIG. 3A). The level of CCL7 in supernatants of EPCs from healthy volunteers and T2DM patients (n=4; FIG. 3B). Western blotting and statistical analyses of CCL7 expression for 2 days in EPCs from healthy volunteers and T2DM patients (n=3; FIG. 3C). Western blotting and statistical analyses of p-eNOS, p-AKT, VEGF, and SDF-1 expressions for 2 days in EPCs from healthy volunteers and T2DM patients (n=3; FIG. 3D). *P<0.05, **P<0.01. n=3 represented EPCs from three different individuals, and each experiment were three independent experiments.

FIGS. 4A to 4D are graphs illustrating the angiogenic factors and the endothelial functions in EPCs from healthy volunteers by the high glucose stimulation were improved via inhibition of CCL7. Western blotting and statistical analyses of CCL7 expression after administration of CCL7 siRNA for 2 days in the high glucose-treated EPCs from healthy volunteers (n=3; FIG. 4A). The tube formation and migration abilities after administration of CCL7 siRNA for 2 days in the high glucose-treated EPCs from healthy volunteers (n=3; FIG. 4B, FIG. 4C) Western blotting and statistical analyses of p-eNOS, p-AKT, VEGF, and SDF-1 expressions after administration of CCL7 siRNA for 2 days in the high glucose-treated EPCs from healthy volunteers (n-3; FIG. 4D). M represents the mannitol treatment. *P<0.05, **P<0.01. n=3 represented EPCs from three different individuals, and each experiment were three independent experiments.

FIGS. 5A to 5D are graphs illustrating the endothelial functions and the angiogenic factors were improved in EPCs from T2DM patients via knockdown of CCL7 by siRNA. Western blotting and statistical analyses of CCL7 expression after administration of CCL7 siRNA for 1 day in EPCs from T2DM patients (n=3; FIG. 5A). The tube formation and migration abilities after administration of CCL7 siRNA for 1 day in EPCs from T2DM patients (n−3; FIG. 5B, FIG. 5C). Western blotting and statistical analyses of p-eNOS, p-AKT, VEGF, and SDF-1 expressions after administration of CCL7 siRNA for 1 day in EPCs from T2DM patients (n=3; FIG. 5D). *P<0.05, **P<0.01. n=3 represented EPCs from three different individuals, and each experiment were three independent experiments.

FIGS. 6A to 6C are graphs illustrating the angiogenic factors and the endothelial functions were improved in EPCs from T2DM patients by treatment with CCL7 neutralizing antibody. The tube formation and migration abilities after administration of CCL7 neutralizing antibody and anti-goat IgG for 1 day in EPCs from T2DM patients (n=3; FIG. 6A, FIG. 6B). Western blotting and statistical analyses of p-eNOS, p-AKT, VEGF, and SDF-1 expressions after administration of CCL7 neutralizing antibody for 1 day in EPCs from T2DM patients (n=3; FIG. 6C). *P<0.05, **P<0.01. n=3 represented EPCs from three different individuals, and each experiment were three independent experiments.

FIGS. 7A to 7E are graphs illustrating the treatment with CCL7 neutralizing antibody improved angiogenesis in type 2 diabetic mice. Foot blood flow monitored by laser Doppler imaging system in each group of mice. Representative evaluation of the ischemic (right) and non-ischemic (left) hind limbs, before, immediately after, and 4 weeks after the surgery. In color-coded images, red indicates normal perfusion and blue indicates a marked reduction in blood flow in the ischemic hind limb. Color-coded images and blood flow ratios (FIG. 7A, FIG. 7B). Circulating EPCs were measured by flow cytometry (FIG. 7C). Anti-CD31 immunostaining showed that CCL7 antibody treatments significantly increased the number of capillaries (FIG. 7D). Angiogenesis in aortic ring cultures from CCL7 antibody-treated mice showed increased number of vessels sprouting (FIG. 7E). Non-DM control group, n=6; DM, n=6; DM+CCL7 antibody 0.1 μg group, n=6; DM+CCL7 antibody 1 μg group, n=6; DM+IgG group, n=4. *p<0.05, **p<0.01 compared with the control group. #p<0.05, ##p<0.01 compared with the DM group.

### DETAILED DESCRIPTION OF THE PREFERRED EMBODIMENTS

The following examples are used to illustrate the present disclosure. A person skilled in the art can easily conceive of the other advantages and effects of the present disclosure based on the invention of the specification. The present disclosure can also be implemented or applied as described in various examples. It is possible to modify or alter the following examples for carrying out the present disclosure without violating its spirit and scope, for different aspects and applications.

It is further noted that the singular forms “a,” “an,” and “the” as used in the present disclosure include plural referents unless expressly and unequivocally limited to one referent. The term “or” is used interchangeably with the term “and/or” unless the context clearly indicates otherwise.

As used herein, the term “comprising” or “comprises” is used in reference to compositions, methods, and respective component(s) thereof, which are essential to the present disclosure, yet open to the inclusion of unspecified elements, whether essential or not.

The present disclosure is directed to a method for preventing or treating peripheral arterial disease (PAD) in a subject in need thereof, comprising administering a effective amount of a chemokine C-C motif ligand 7 (CCL7) antagonist to the subject, in which the CCL7 antagonist is capable of inhibiting CCL7 activity. In one embodiment of the present disclosure, the CCL7 antagonist, also known as monocyte-specific chemokine (MCP)-3 antagonist, is capable of preventing the binding of CCL7 to its receptor. In another embodiment, the CCL7 antagonist is an agent that inhibits intracellular signaling generated by the binding of CCL7 to its receptor. For example, the CCL7 antagonist may be directed against at least one of CCL7 and the receptor of CCL7, thereby blocking the CCL7 signaling. As used herein, the receptor of CCL7 includes, but is not limited to, CCR1, CCR2, CCR3, CCR5.

As used herein, the terms “CCR1” or “CCR1 receptor”, “CCR2” or “CCR2 receptor”, “CCR3” or “CCR3 receptor”, and “CCR5” or “CCR5 receptor” are used interchangeably and have their general meaning in the art. The CCR1, CCR2, and CCR3 receptors may be from any source but typically a mammalian (e.g., human or non-human primate) CCR1, CCR2, CCR3, and CCR5 receptor. In some embodiments of the present disclosure, the CCR1, CCR2, CCR3 and CCR5 receptors are human receptors.

As used herein, the term “CCL7 ” has its general meaning in the art. CCL7 is a natural ligand of the CCR3 receptor and may be from any source, but typically is a mammalian (e.g., human or non-human primate) CCL7. In one embodiment of the present disclosure, the CCL7 is a human CCL7.

As used herein, the term “CCL7 antagonist” includes any entity that, upon administration to a subject, results in inhibition or down-regulation of a biological activity associated with CCL7 in the subject, including any of the downstream biological effects otherwise resulting from the binding of CCL7 to its receptor. The CCL7 antagonist includes any agent that may inhibit CCL7 activity or block activation of the receptor of CCL7 or any of the downstream biological effects of activation of the receptor of CCL7. Such a CCL7 antagonist includes any agent that is able to interact with CCL7 so that its normal biological activity is prevented or reduced. For example, said agent may be a small organic molecule or an antibody directed against CCL7, such as a CCL7 neutralizing antibody, which can block the interaction between CCL7 and its receptor, or which can block the activity of CCL7. The CCL7 antagonist may also be a small molecule or an antibody directed against the receptor of CCL7, which may act by occupying the ligand binding site or a portion thereof of the receptor, thereby making the receptor inaccessible to its ligand, CCL7.

As used herein, the terms “PAD,” “peripheral arterial disease,” or “peripheral artery disease” refer to a condition in which narrowed arteries reduce blood flow to the arms or legs. In particular, the legs or arms cannot receive enough blood flow to keep up with demand, which causes leg pain when walking and other symptoms such as claudication and/or restpain. In addition, peripheral artery disease may be regarded as a sign of a buildup of fatty deposits in the arteries, that is, atherosclerosis that causes narrowing of the arteries that can reduce blood flow in the legs.

In one embodiment of the present disclosure, the subject suffering from PAD may also have limb ischemia, diabetic ulcer, gangrene, intermittent claudication, thromboangiitis obliterans (Buerger's disease), Raynaud's disease, and/or vasculitis, but is not limited thereto. In another embodiment, the subject to be treated by the method of the present disclosure suffers from diabetes, chronic arterial occlusion, vascular spasm, scleroderma, or vasculitis.

In patients with PAD, the number of endothelial progenitor cells (EPCs) is reduced, and the function of EPCs, as defined by colony-forming capacity and migratory activity, is significantly reduced and associated with reduced neovascularization in hindlimb ischemia. Similarly, the number of EPCs is reduced in patients with type I or type II diabetes. The reduction in the number of EPCs and their function is associated with some vascular complications, such as endothelial dysfunction, that predispose patients to impaired neovascularization after ischemic events.

The methods and the CCL7 antagonist of the present disclosure may be used to treat a variety of conditions that would benefit from stimulation of angiogenesis, stimulation of vasculogenesis, increased blood flow, and/or increased vascularity.

As used herein, the term “angiogenesis” refers to the growth or formation of blood vessels. Angiogenesis includes the growth of new blood vessels from pre-existing vessels, as well as vasculogenesis, which refers to the spontaneous formation of blood vessels, and intussusception, which refers to the formation of new blood vessels by splitting off existing blood vessels. Angiogenesis includes “neovascularization,” “regeneration of blood vessels,” “generation of new blood vessels,” and “revascularization.

As used herein, the term “treating” or “treatment” refers to achieving a desired pharmacological and/or physiological effect, such as stimulation of angiogenesis. The effect may be prophylactic in the sense of preventing, in whole or in part, a disease or a symptom thereof, or therapeutic in the sense of curing, mitigating, relieving, remedying, or ameliorating, in whole or in part, a disease or an adverse effect attributable to a disease.

As used herein, the terms “symptoms associated with PAD”, “symptoms resulting from ischemia” and “symptoms caused by ischemia” refer to symptoms that include impaired or loss of organ function, cramping, claudication, numbness, tingling, weakness, pain, reduced wound healing, inflammation, skin discoloration and gangrene. As used herein, “Treatment” means any treatment of a disease or condition and includes: (a) preventing a disease or condition from occurring (e.g., preventing the loss of a skin graft or reattached limb due to inadequate blood flow) in a subject who may be predisposed to, but has not yet been diagnosed with, the disease; (b) inhibiting the disease or symptom thereof, such as inhibiting the disease or symptom thereof, e.g., slowing or arresting its development; or (c) ameliorating the disease or symptom thereof (e.g., enhancing the development of neovascularization around an ischemic tissue to improve blood flow to the tissue). In the context of the present disclosure, stimulation of angiogenesis is used for a subject having a disease or condition amenable to treatment by increasing vascularity and increasing blood flow. Such a subject may be identified by a healthcare professional based on the results of any suitable diagnostic method.

As used herein, the terms “patient” and “subject” are used interchangeably. The term “subject” means a human being or an animal. Examples of a subject include, but are not limited to, humans, monkeys, mice, rats, woodchucks, ferrets, rabbits, hamsters, cows, horses, pigs, deer, dogs, cats, foxes, wolves, chickens, emu, ostrich, and fish. In certain embodiments of the present disclosure, the subject is a mammal, e.g., a primate, such as a human.

As used herein, the phrase “an effective amount” refers to the amount of an active agent (e.g., CCL7 antagonist) that is required to confer a desired therapeutic effect (e.g., a desired level of angiogenic stimulation) in the treated subject. Effective doses will vary, as recognized by those skilled in the art, depending on the routes of administration, the use of excipients, the possibility of co-administration with other therapeutic treatments, and the condition to be treated.

In one embodiment of the present disclosure, the effective amount of the CCL7 antagonist is from about 0.01 μg/kg to about 100 mg/kg, such as from about 0.05 μg/kg to about 90 mg/kg, from about 0.1 μg/kg to about 80 mg/kg, from about 0.2 μg/kg to about 70 mg/kg, from about 0.4 μg/kg to about 60 mg/kg, from about 0.6 μg/kg to about 50 mg/kg, from about 0.7 μg/kg to about 40 mg/kg, from about 0.8 μg/kg to about 30 mg/kg, from about 0.9 μg/kg to about 20 mg/kg from about 1 μg/kg to about 10 mg/kg, from about 1.5 μg/kg to about 5 mg/kg, from about 2 μg/kg to about 1 mg/kg, from about 2.5 μg/kg to about 500 μg/kg, from about 3 μg/kg to about 400 μg/kg, from about 3.5 μg/kg to about 300 μg/kg, from about 4 μg/kg to about 200 μg/kg, from about 4.5 μg/kg to about 100 μg/kg, from about 5 μg/kg to about 50 μg/kg, from about 5.5 μg/kg to about 40 μg/kg, from about 6 μg/kg to about 30 μg/kg, from about 6.5 μg/kg to about 20 μg/kg, from about 7 μg/kg to about 10 μg/kg, from about 7.5 μg/kg to about 9.5 μg/kg, or from about 8 μg/kg to about 9 μg/kg. In another embodiment, the effective amount of the CCL7 antagonist has a lower limit chosen from 0.01 μg/kg, 0.05 μg/kg, 0.1 μg/kg, 0.5 μg/kg, 1 μg/kg, 2 μg/kg, 3 μg/kg, 4 μg/kg, and 5 μg/kg, and an upper limit was chosen from 100 mg/kg, 90 mg/kg, 80 mg/kg, 70 mg/kg, 60 mg/kg, 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 10 μg/kg, 9 μg/kg, 8 g/kg, 7 μg/kg, and 6 μg/kg.

In one embodiment of the present disclosure, the CCL7 antagonist is administered 1 to 2 times over a period of 2 to 4 days. In another embodiment, the CCL7 antagonist is administered 8 to 15 times over a period of 3 to 5 weeks. For example, the CCL7 antagonist is administered 3 times over a week, or 10 times over a period of 4 weeks. In yet another embodiment, the CCL7 antagonist is administered 1-4 weeks apart, such as one week, 8 days, 9 days, 10 days, 11 days, 12 days, 13 days, two weeks, 15 days, 16 days, 17 days, 18 days, 19 days, 20 days, three weeks, 22 days, 23 days, 24 days, 25 days, 26 days, 27 days, or four weeks.

As used herein, the term “administering” or “administration” refers to the placement of an active agent (e.g., CCL7 antagonist) in a subject by a method or route that results in at least partial localization of the active agent to a desired site such that a desired effect is produced. The active agent described herein may be administered by any appropriate route known in the art including, but not limited to, oral or parenteral routes, including intraperitoneal, intravenous, intradermal, intramuscular, subcutaneous, or transdermal routes.

In one embodiment of the present disclosure, the CCL7 antagonist may be formulated into a pharmaceutical composition to be administered to the subject. In certain embodiments, the present disclosure provides a pharmaceutical composition for stimulating angiogenesis, comprising the CCL7 antagonist and a pharmaceutically acceptable carrier thereof. The pharmaceutical composition provided in the present disclosure may efficiently prevent or treat PAD and/or peripheral ischemic tissue or tissue damaged by peripheral ischemia.

In one embodiment of the present disclosure, the pharmaceutically acceptable carrier may be a diluent, a disintegrant, a binder, a lubricant, a glidant, a surfactant, or a combination thereof.

In one embodiment of the present disclosure, the pharmaceutical composition is a sterile injectable composition which may be a solution or suspension in a non-toxic, parenterally acceptable vehicle or solvent. Acceptable vehicles and solvents that may be used include 1,3-butanediol, mannitol, water, Ringer's solution, and isotonic sodium chloride solution. In addition, solid oils (e.g., synthetic mono-or di-glycerides) are commonly used as solvents or suspending agents. Fatty acids such as oleic acid and its glyceride derivatives are useful in the preparation of injectables, as are natural pharmaceutically acceptable oils such as olive oil and castor oil, especially in their polyoxyethylated versions. These oil solutions or suspensions may also contain a long chain alcohol diluent or dispersant, carboxymethyl cellulose or similar dispersants. Other commonly used surfactants such as Tweens and Spans or other similar emulsifiers or bioavailability enhancers commonly used in the manufacture of pharmaceutically acceptable solid, liquid or other dosage forms may also be used for formulation purposes.

The carrier in the pharmaceutical composition must be “acceptable” in the sense that it is compatible with the active agent of the composition (and can be capable of stabilizing the active agent) and not deleterious to the subject to be treated. One or more solubilizing agents can be utilized as pharmaceutical excipients for the delivery of an active compound. Examples of other excipients or carriers include colloidal silicon oxide, magnesium stearate, cellulose, and sodium lauryl sulfate.

Many examples have been used to illustrate the present disclosure. The examples below should not be taken as a limit to the scope of the present disclosure.

### MATERIALS AND METHODS

### Cell Culture of Human Aortic Endothelial Cells (HAECs)

Human aortic endothelial cells (HAECs; ScienCell Research Laboratories, Faraday Ave, Carlsbad CA, USA) were cultured with endothelial cell medium (ScienCell Research Laboratories, 1001, Faraday Ave, Carlsbad CA, USA) containing VEGF and 1% penicillin/streptomycin and with 5% FBS. Cultured dish was coated with fibronectin before being used. Cells (2×106) were maintained in an atmosphere of 95% air and 5% CO2 at 37° C. Cells were treated with mannitol or the high glucose (25 mM, Sigma-Aldrich, St. Louis, Missouri, USA) for 2 days.

### Cell Culture of EPCs

Total mononuclear cells (MNCs) were isolated from peripheral blood of healthy volunteers or patients with T2DM by density gradient centrifugation with Histopaq-1077 (density 1.077 g/ml; Sigma-Aldrich, 10771, St. Louis, Missouri, USA). Total MNCs (5×106) were plated in 2 mL endothelial cell basal medium (EBM-2; Lonza, CC-3156, Basel, Switzerland), with supplements (hydrocortisone, hFGF-B, VEGF, R3-IGF-1, ascorbic acid, hEGF, GA-1000) and 20% fetal bovine serum on fibronectin-coated six-well plates at 37° C. in a 5% CO2 incubator. Under daily observation, after 4 days of culturing, medium was changed and nonadherent cells were removed. EPCs were emerged 2-4 weeks after the start of the culture of MNCs. EPCs in the shape of cobblestone, and this kind of shape is typical monolayer growth pattern of mature endothelial cells. Some EPCs from T2DM patients were treated with 1 or 10 ng/mL human CCL7 antibody (R&D Systems, MAB282, Minnesota, USA). The protocol of clinical study was approved by the Institutional Review Board (IRB) of Taipei Veterans General Hospital, Taipei, Taiwan, ROC (no. 2021-02-010AC). The investigation conformed to the principles outlined in the Declaration of Helsinki.

### Investigations of Signaling Pathway

Some cells were treated with 1 or 10 ng/ml recombinant human CCL7 protein (R&D Systems, 282-P3, Minnesota, USA) for 2 days. To investigate the CCL7 and AKT signaling pathway, cells were pre-treated with 3 μM LY294002 (Cayman Chemical Company, No. 70920, Ann Arbor, MI, USA), an AKT inhibitor, then treated with 10 ng/mL recombinant human CCL7 protein. To investigate the CCL7 and ERK signaling pathway, cells were pre-treated with 10 μM U0126 (Cayman Chemical Company, No. 70970, Ann Arbor, MI, USA), an ERK inhibitor, then treated with 10 ng/mL recombinant human CCL7 protein. To investigate the CCL7 and HO-1 signaling pathway, cells were pre-treated with 1, 50 or 100 μM CORM-3 (Tocris Bioscience, No. 5320, Bristol, UK), an HO-1 inducer, then treated with 10 ng/mL recombinant human CCL7 protein. To investigate the CCL7 and CCR3, cells were pre-treated with 2 or 20 nM SB328437 (Tocris Bioscience, No. 3650, Bristol, UK), an CCR3 antagonist, then treated with 10 ng/ml recombinant human CCL7 protein.

### Transfection of CCL7 siRNA

Cells were cultured in 6-well plates for 24 hours. CCL7 siRNA (Santa Cruz Biotechnology, sc-72035, Dallas, TX, USA) with an equivalent siRNA concentration of 100 nM were then transfected into the cells by oligofectamine (Thermo Fisher Scientific, 12252011, Waltham, MA, USA) and incubated for 5 hours. After transfection, the reagent was replaced by cultured medium and cultured for 48 hours.

### Migration Assay

The scratch-wound assay was used to evaluate the migration ability of cells. Cells were seeded in each well of a 6-well plate and allowed to grow to 80-90% confluence. The media was removed and the confluent cell sheet was wounded through scratching the culture well surface by p200 tip. Photos of the scratch were acquired at the different timing after first scratch. Gap distance was evaluated by the computer software, ImageJ.

### Tube Formation Assay

In vitro tube formation assay was performed with an angiogenesis assay kit (Merck Millipore, ECM625, Darmstadt, Germany). ECMatrix gel solution were mixed with ECMatrix diluent buffer and placed in a 96-well plate. EPCs or HAECs were collected by trypsinization, and 2×104 cells/well were seeded into ECMatrix gel in 96 well in 100 μL cultured medium with 10% FBS for 6˜8 hours at 37°° C. and 5% CO2. Tube formation was inspected under an inverted light microscope (×40). Three representative fields were taken, and the average of the total area of complete tubes formed by cells were compared using computer software, ImageJ.

### Western Blot Analysis

Equal amounts of protein were extracted using lysis buffer, and proteins were separated to SDS-PAGE on 4-12% gradient gels. After electrophoresis (Bio-Rad Laboratories, Hercules, CA, USA), the proteins were transferred onto PVDF membranes. Membranes were incubated with primary antibodies against CCL7 (R&D Systems, MAB282; Minnesota, USA), phospho-eNOS (Cell Signaling, 9571S; Boston, MA, USA), eNOS (Cell Signaling, 32027S; Boston, MA, USA), phospho-AKT (BD Biosciences, 550747; NJ, USA), AKT (BD Biosciences, 610868; NJ, USA), VEGF (Santa Cruz Biotechnology, sc-152; Dallas, TX, USA), SDF-1 (Cell Signaling, 3530S; Boston, MA, USA), phospho-ERK (Cell Signaling, 9106S; Boston, MA, USA), ERK (Cell Signaling, 9102S; Boston, MA, USA), CCR3 (Thermo Fisher Scientific, #PA5-19859, Waltham, MA, USA), NRF2 (Cell Signaling, 12721S; Boston, MA, USA), HO-1 (Cell Signaling, 70081S; Boston, MA, USA), TNF-a (Cell Signaling, 3707S; Boston, MA, USA), IL-6 (Cell Signaling, 12153S; Boston, MA, USA), IL-1β (Santa Cruz Biotechnology, sc-7884; Dallas, TX, USA), phosphor-p65 (Cell Signaling, 3031S; Boston, MA, USA), p65 (BD Biosciences, 610868; NJ, USA) at 4° C. overnight. After washing three times, the membranes were incubated with appropriate secondary antibodies (1:1000) for 1 hour at room temperature. Finally, the membranes were visualized by using ECL kit. The expressions of these protein were normalized to expression of β-actin (Merck, 3423208, Darmstadt, Germany).

### ELISA

Plasma concentration of CCL7 in T2DM patients and normal subject and primary cell medium concentration of CCL7 were analyzed by human CCL7 /MCP-3 quantikine ELISA kit (R&D, DCCL700, Minneapolis, MN, USA). The supernatant of HAECs (2×106) were stimulated for 3 days with or without 25 mM the high glucose. The supernatant of EPCs (5×106) from healthy volunteers or patients with T2DM were incubated for 3 days. Methods were used following the instructions provided by the original manufacturer.

### Transfection of the eNOS Gene

A red fluorescent protein (RFP)-tagged bovine wild type (WT)-eNOS gene was purchased from Addgene (plasmid #22497, Cambridge, MA, USA). HAECs grown to 80% confluence in 6-well plate were transfected with 500 ng of the WT-eNOS using lipofectamine 2000 reagent (Invitrogen, 52887, Carlsbad, CA, USA) according to the manufacturer's instructions. After incubation for 4 hours at 37° C., lipofectamine mixtures were washed out, and the reagent was replaced by cultured medium and cultured for 24 hours.

### Immunoprecipitation (IP)

HAECs were incubated with CCL7 recombinant protein for 30 minutes. Cells were chilled on ice and cell extracts prepared with lysis buffer (1% Triton X-100, 2.5 mM EDTA, 25 mM Tris-HCl, 150 mM NaCl, 5% glycerol, 1 mM PMSF, pH 7.4). Lysates were cleared by centrifugation at 13000 rpm for 30 minutes and incubated with CCR3 polyclonal antibody (Thermo Fisher Scientific, #PA5-117846, Waltham, MA, USA) attached to agarose beads overnight at 4°° C. on a rocking. Beads were then collected by centrifugation at 10000 rpm for 3 minutes at 4° C., extensively washed in lysis buffer. The proteins were separated on a 12% SDS-polyacrylamide gel, transferred to a PVDF membrane, and analyzed by immunoblotting with the corresponding antibodies.

### Statistical Analysis

The results were presented as the mean±standard deviation (SD). Statistical analyses were performed by using unpaired Student's t test. P value<0.05 was considered statistically significant.

### Example 1: CCL7 Levels in the Supernatants of HAECs

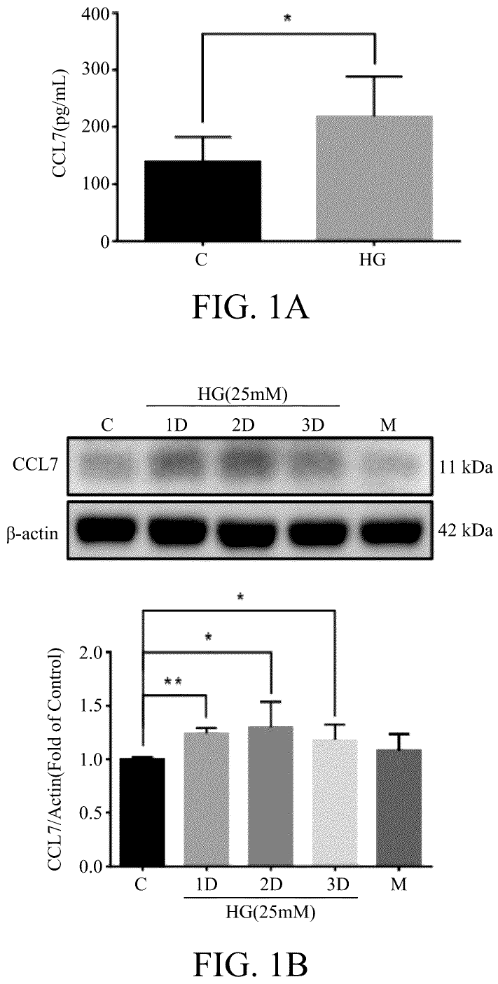

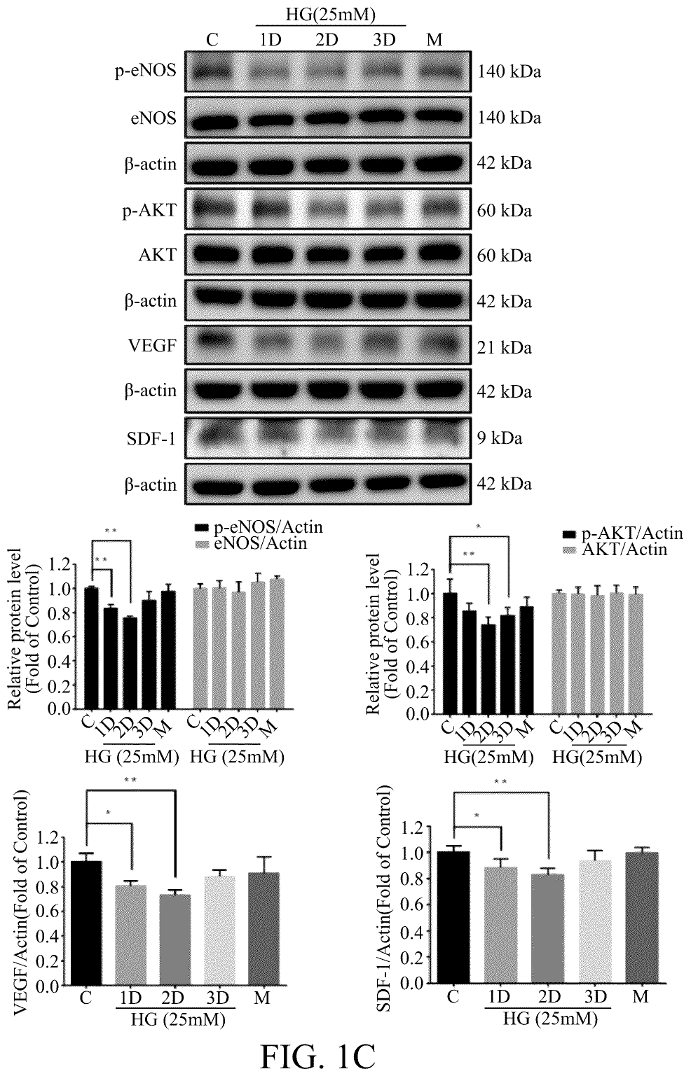

The concentrations of CCL7 in primary cell medium of HAECs under the high glucose conditions were significantly elevated in comparison with control group (FIG. 1A). In the present disclosure, mannitol was used as a control for osmolarity. CCL7 expression was significantly increased in HAECs under the high glucose conditions (FIG. 1B). The protein expressions of p-eNOS, VEGF and SDF-1 were decreased in HAECs, which were incubated with the high glucose for 1 day or 2 days. The protein expressions of p-AKT was reduced in HAECs by treatment with the high glucose for 2 days or 3 days (FIG. 1C). These results indicated that CCL7 expression was induced and angiogenic factors were reduced under the stimulation of the high glucose for 2 days in HAECs. In light of the above results shown in FIGS. 1A-1C, CCL7 expression was increased and the angiogenic factors expressions were decreased in HAECs under the high glucose conditions.

### Example 2: Effects of Knockdown of CCL7 by siRNA in HAECs

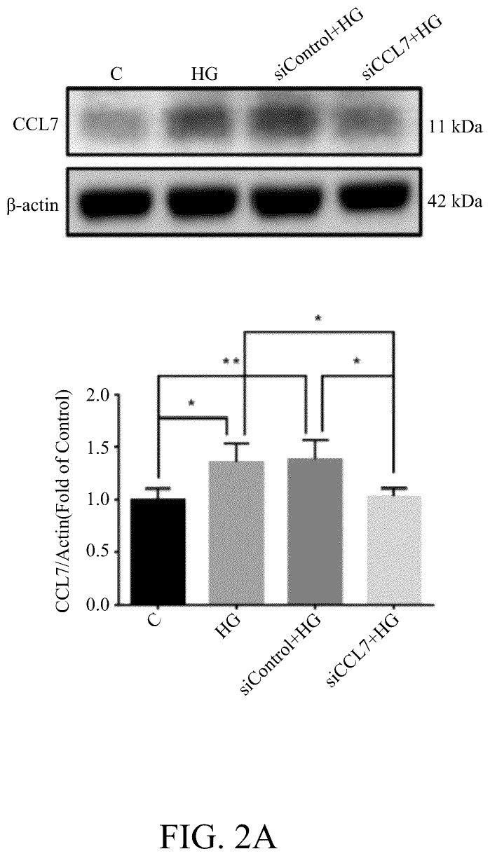

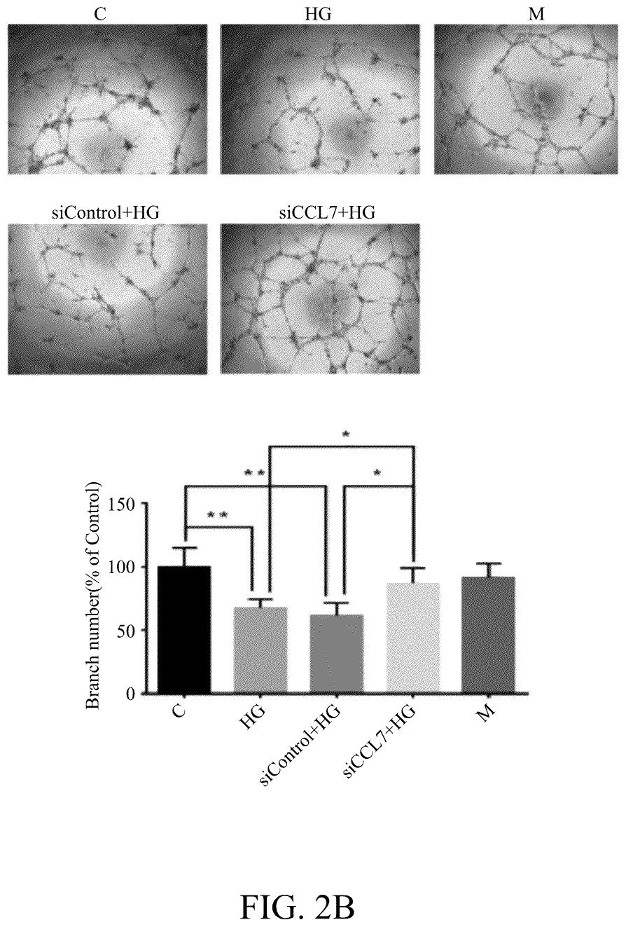

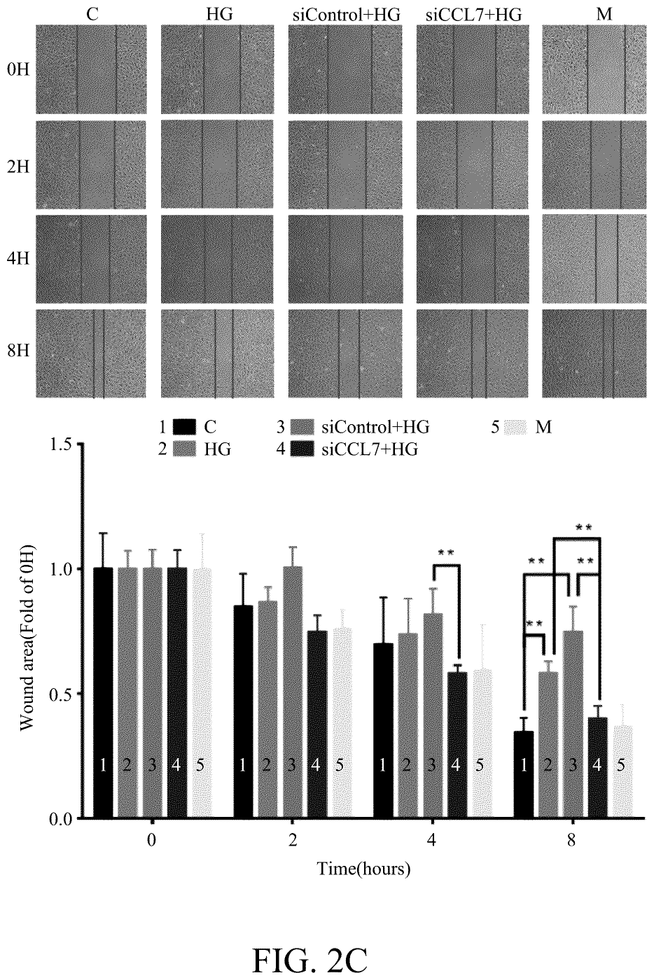

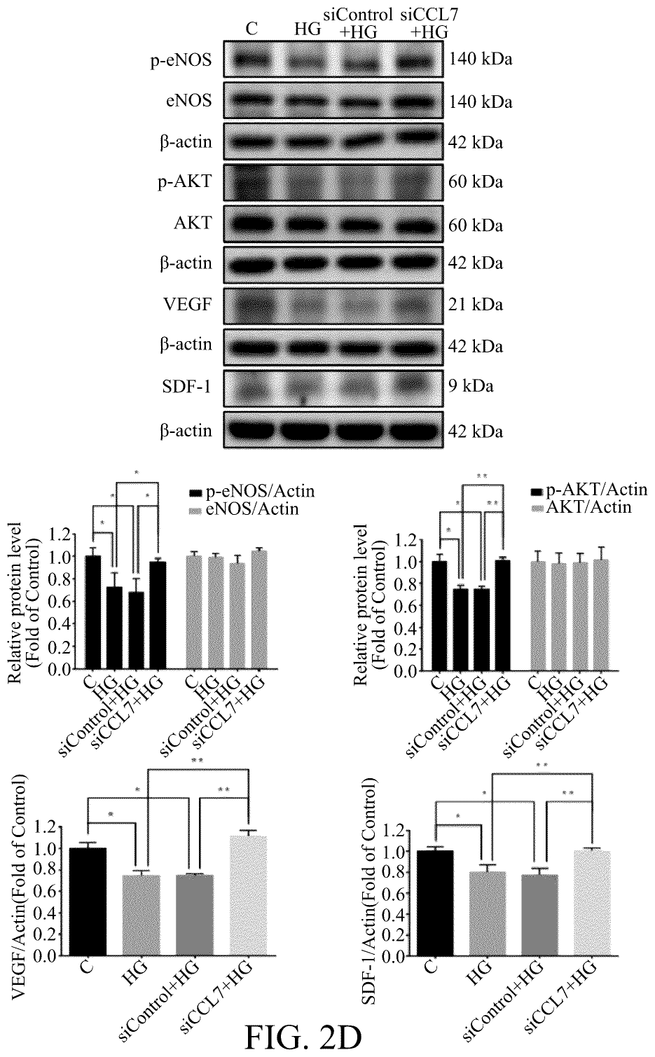

As can be seen in FIG. 2A, decreased expression of CCL7 was observed in HAECs via knockdown of CCL7 by siRNA. In addition, knockdown of CCL7 by siRNA improved the high glucose-impaired tube formation in HAECs (FIG. 2B), and knockdown of CCL7 by siRNA promoted the migration ability in the high glucose-stimulated of HAECs in comparison with the high glucose group (FIG. 2C). Meanwhile, the protein expressions of the angiogenic factors including p-eNOS, p-AKT, VEGF and SDF-1 were also increased via knockdown of CCL7 by siRNA in HAECs under the high glucose conditions (FIG. 2D). According to these results, inhibition of CCL7 increased protein expression of angiogenic factors and repaired impaired migration and tube formation ability in HAECs under the high glucose conditions. Accordingly, it was demonstrated that knockdown of CCL7 by siRNA improved the angiogenic factors and the endothelial functions in HAECs under the high glucose conditions.

### Example 3: CCL7 Levels in EPCs from T2DM Patients

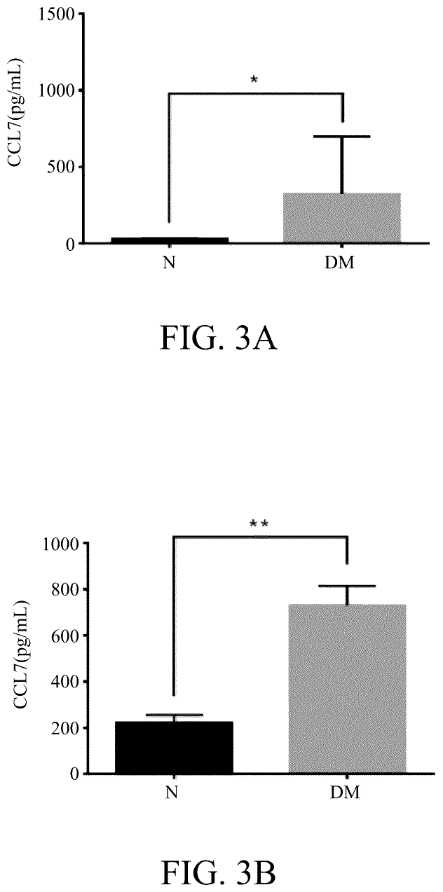

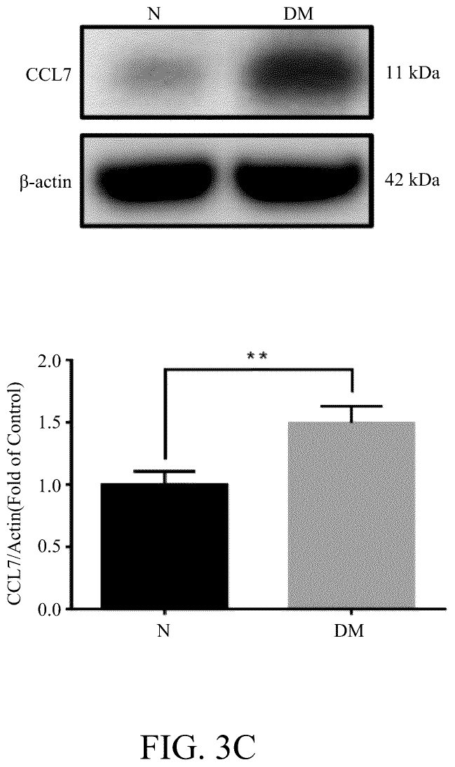

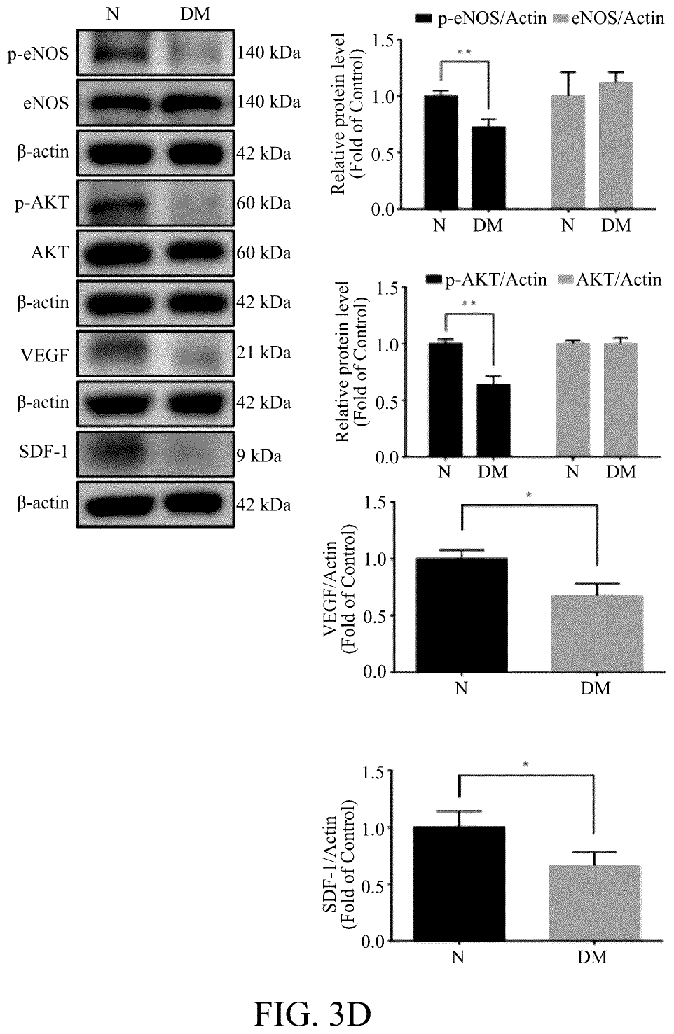

The levels of CCL7 in plasma were significantly higher from T2DM patients in comparison with healthy volunteers (FIG. 3A). The concentrations of CCL7 in primary cell medium of EPCs from T2DM patients were significantly elevated in comparison with healthy volunteers (FIG. 3B). Expression of CCL7 was increased in EPCs from T2DM patients compared to healthy volunteers (FIG. 3C). Moreover, the protein expressions of the angiogenic factors in EPCs from T2DM patients and healthy volunteers were examined. Meanwhile, the angiogenic factors, including p-eNOS, p-AKT, VEGF, and SDF-1, were lower in EPCs from T2DM patients compared to healthy volunteers (FIG. 3D). These data indicated that CCL7 was increased, and the expressions of the angiogenic factors were lower in EPCs from T2DM patients.

### Example 4: Effects of Knockdown of CCL7 by siRNA in EPCs from Healthy Volunteers Under the High Glucose Conditions

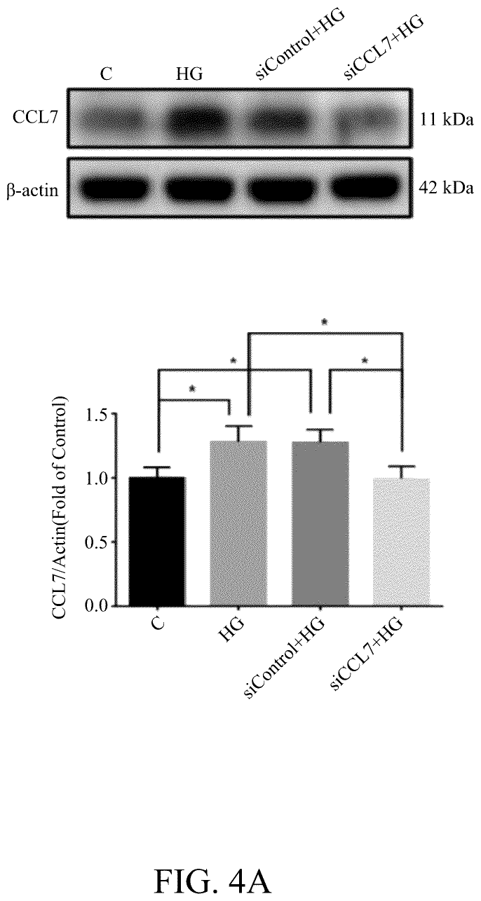

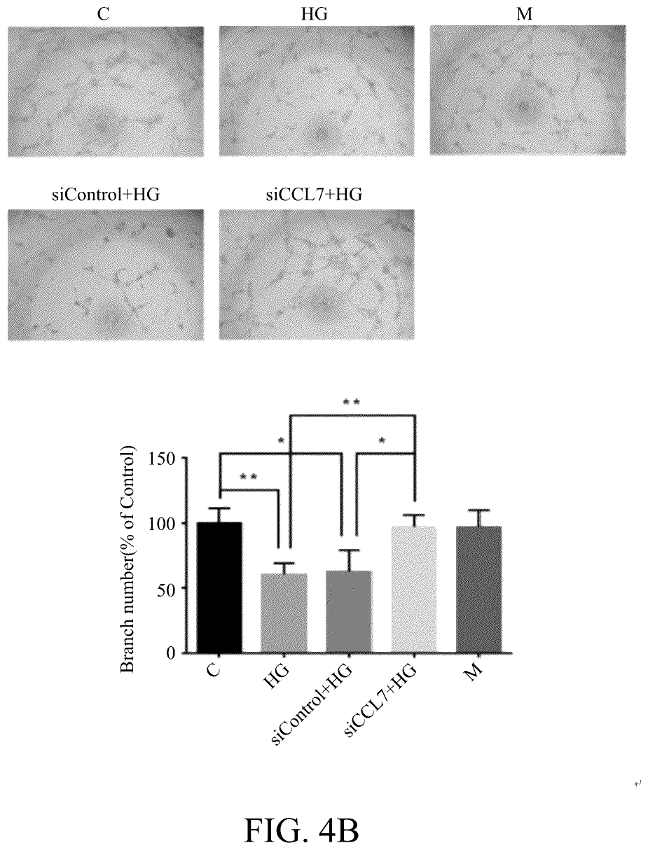

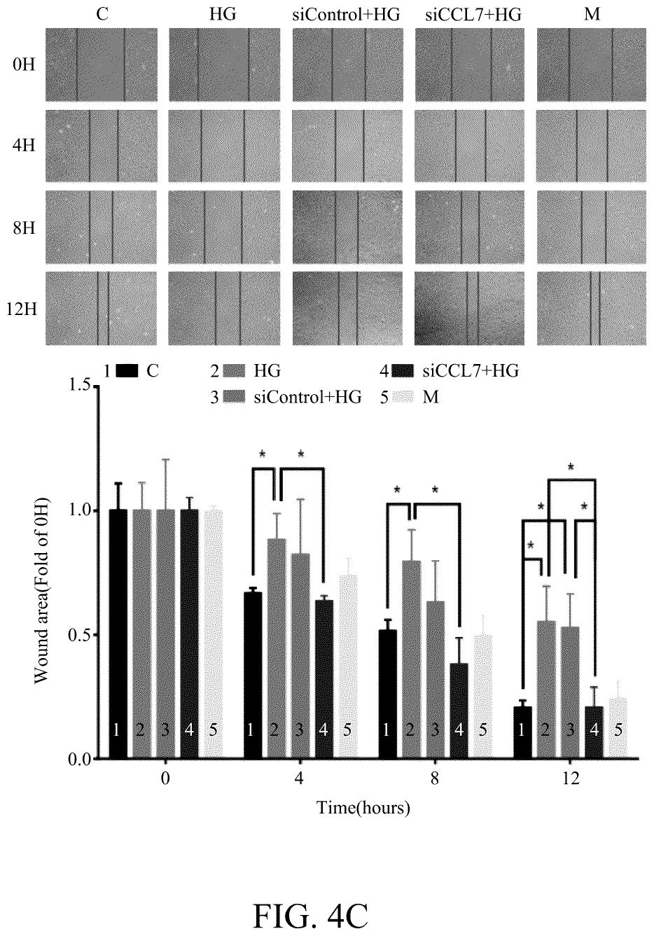

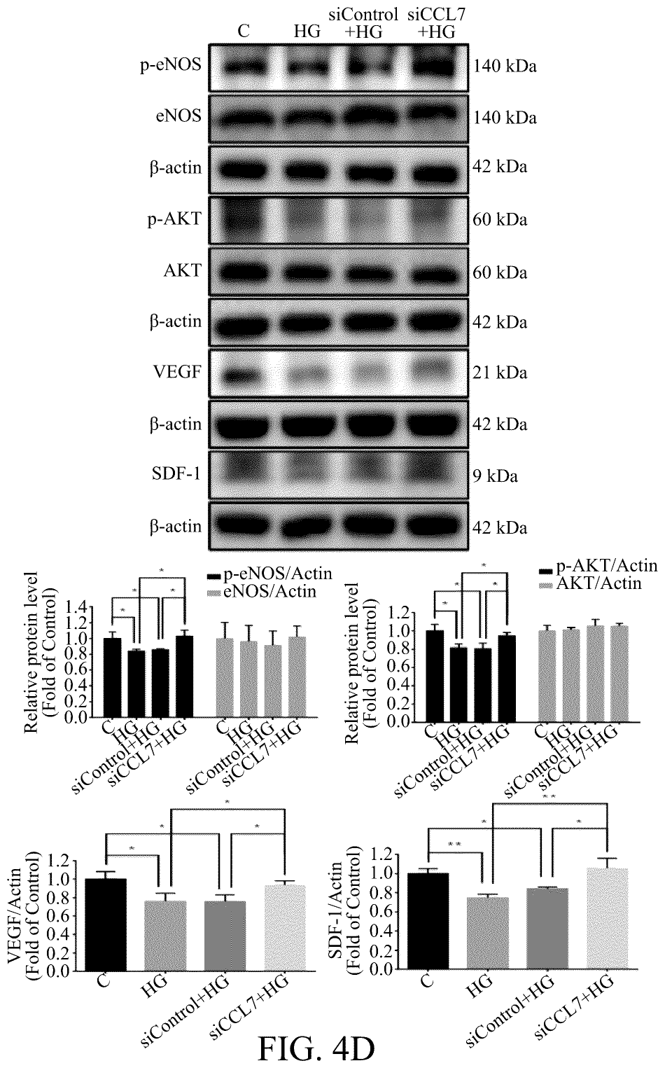

First, inhibition of the high glucose-induced CCL7 expression was observed in EPCs from healthy volunteers via knockdown of CCL7 by siRNA (FIG. 4A). Knockdown of CCL7 by siRNA improved the ability of tube formation under the high glucose conditions (FIG. 4B). In addition, knockdown of CCL7 by siRNA promoted the migration ability in EPCs from healthy volunteers under the high glucose conditions in comparison with the high glucose group (FIG. 4C). Meanwhile, the protein expressions of the angiogenic factors also increased via knockdown of CCL7 by siRNA in EPCs from healthy volunteers under the high glucose conditions (FIG. 4D). According to these results, inhibition of CCL7 improved protein expression of angiogenic factors and repaired injured angiogenesis and migration ability in EPCs from healthy volunteers under the high glucose conditions.

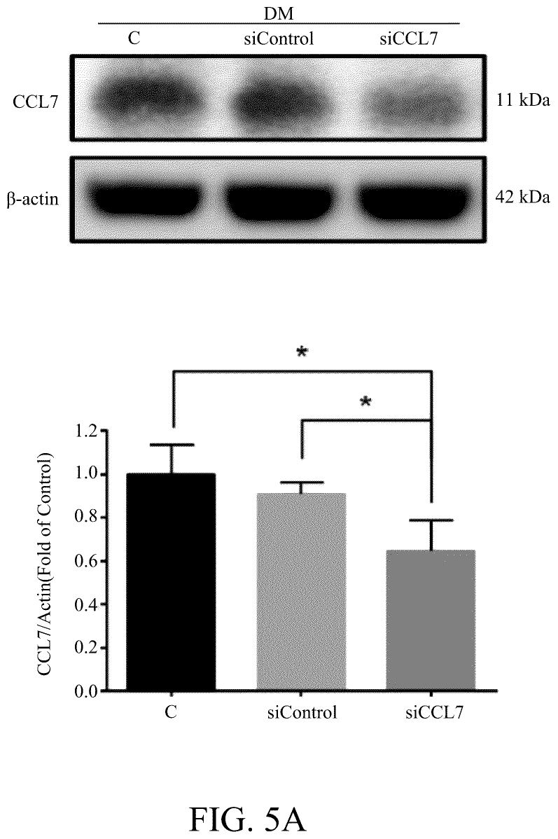

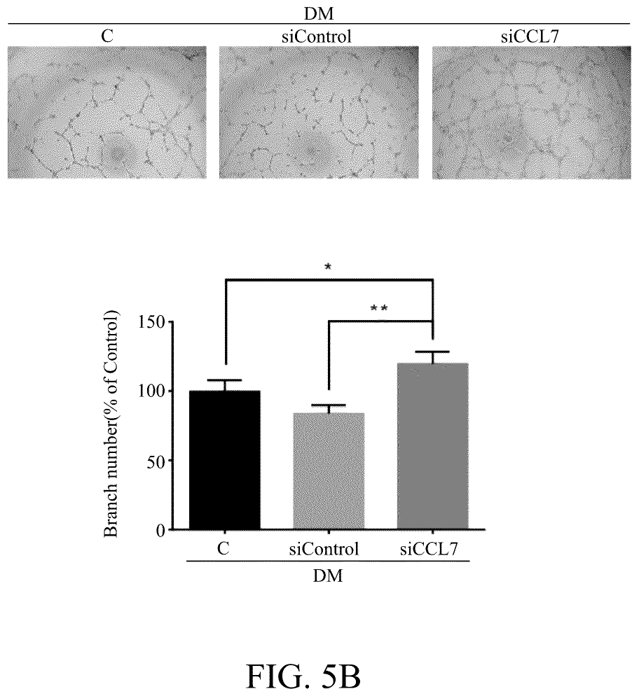

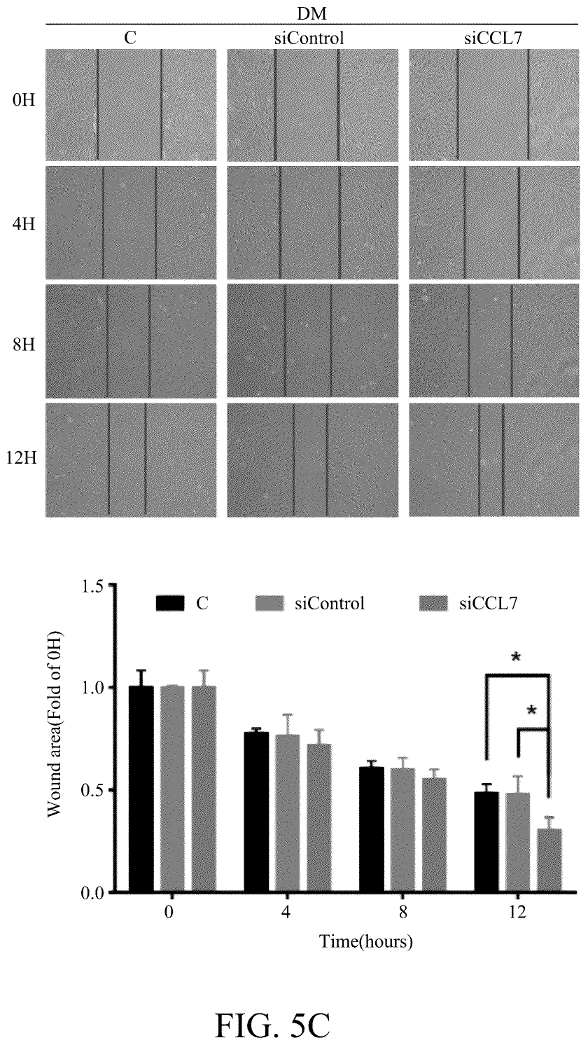

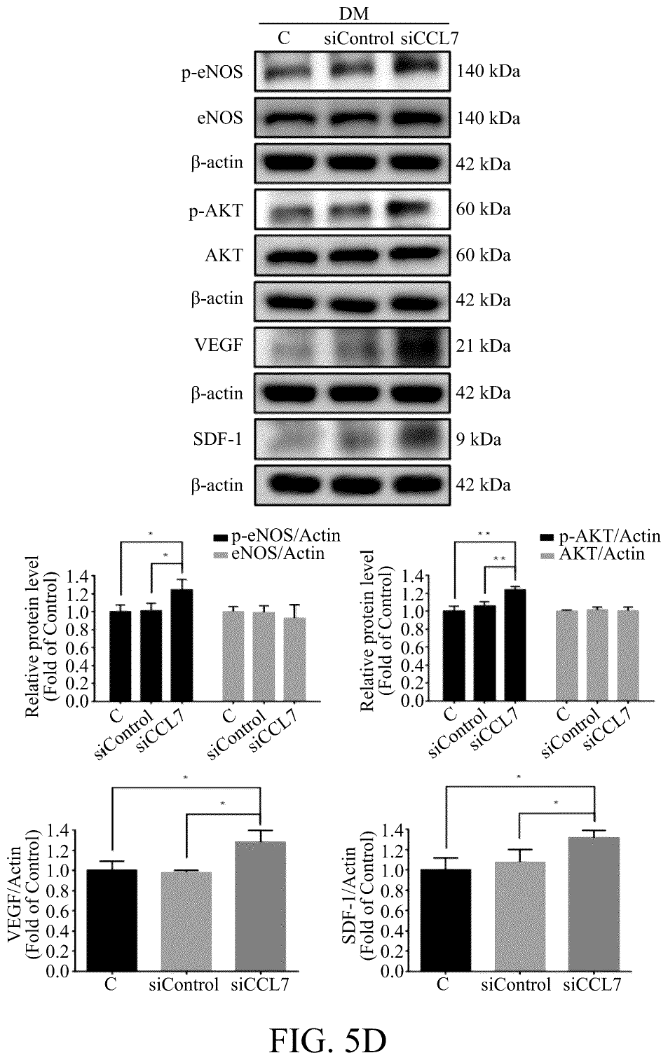

Moreover, inhibition of CCL7 expression was observed in EPCs from T2DM patients via knockdown of CCL7 by siRNA (FIG. 5A). Next, we wanted to confirm whether knockdown of CCL7 by siRNA could repair impaired endothelial dysfunction of EPCs from T2DM patients. The ability of tube formation was improved in EPCs from T2DM patients via knockdown of CCL7 by siRNA (FIG. 5B). In addition, knockdown of CCL7 by siRNA promoted the migration ability in EPCs from T2DM patients (FIG. 5C). Meanwhile, the protein expressions of the angiogenic factors increased via knockdown of CCL7 by siRNA in EPCs from T2DM patients (FIG. 5D). Therefore, these results indicated that inhibition of CCL7 increased p-AKT, p-eNOS, VEGF and SDF-1 protein expressions and promoted angiogenesis and migration ability of EPCs from T2DM patients.

### Example 5: Treatment with CCL7 Neutralizing Antibody in EPCs from T2DM Patients

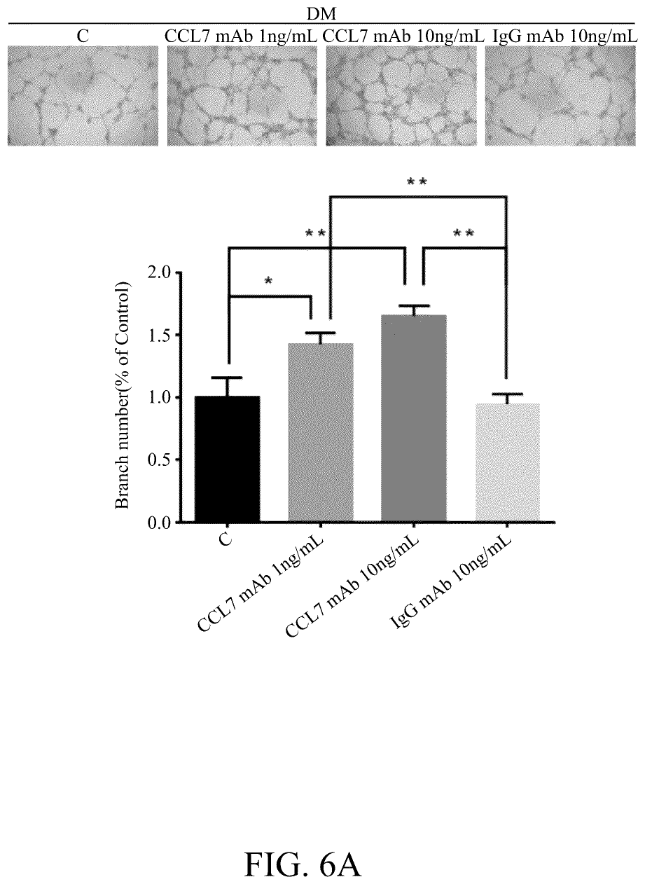

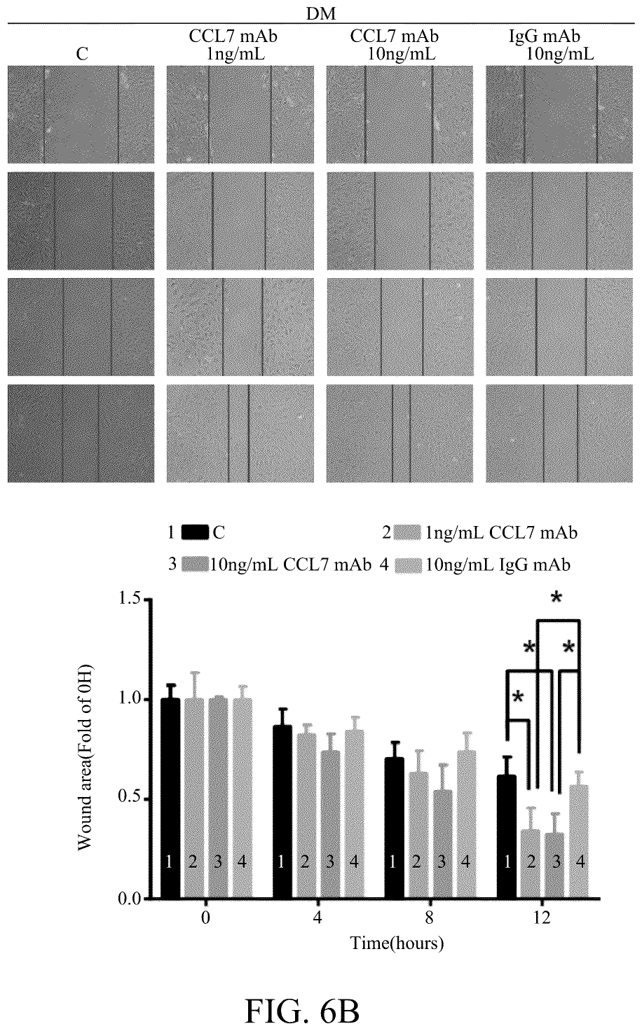

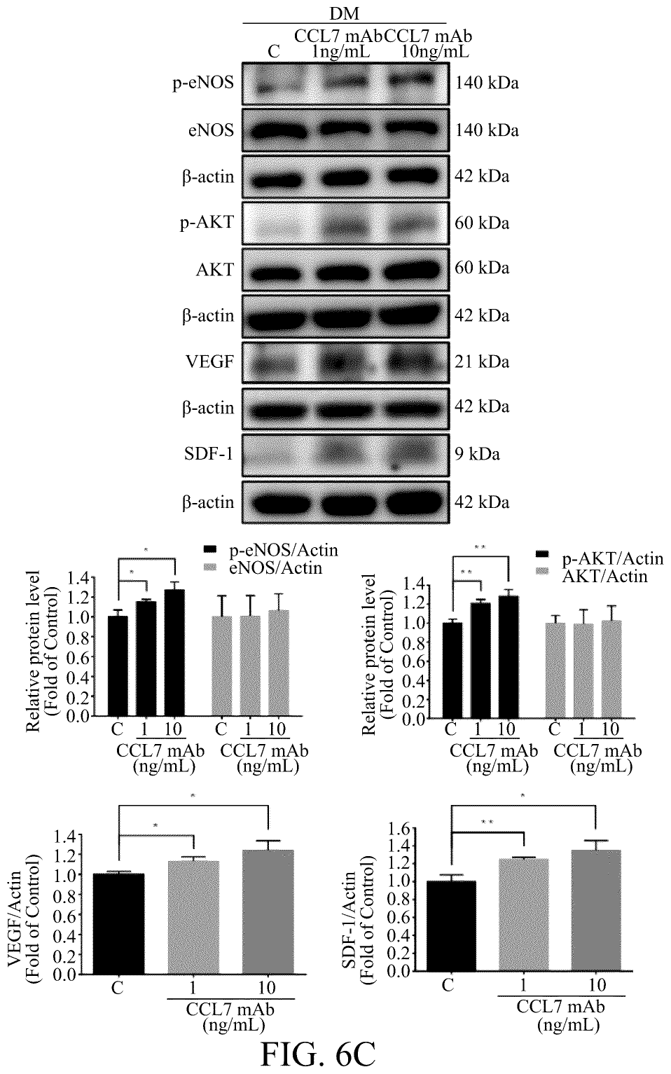

Administration of CCL7 neutralizing antibody improved the ability of tube formation (FIG. 6A). Moreover, treatment with CCL7 antibody promoted the migration ability in EPCs from T2DM patients (FIG. 6B). At the same time, the protein expressions of the angiogenic factors increased by treatment with CCL7 neutralizing antibody in EPCs from T2DM patients (FIG. 6C). Therefore, these results showed that inhibition of CCL7 increased the protein expressions of the angiogenic factors and elevated angiogenesis and migration ability of EPCs from T2DM patients.

Based on the results shown in Examples 1-5 and FIGS. 1-7, CCL7, one of the inflammatory chemokines, was significantly enhanced in EPCs from T2DM or in HAECs under the high glucose conditions. The endothelial functions and the levels of the angiogenic factors were improved in HAECs and EPCs under the high glucose conditions by inhibition of CCL7. CCL7 reduced the expression of angiogenic factors and directly impaired endothelial function in HAECs and EPCs from healthy volunteers. Moreover, CCL7 caused endothelial dysfunction through CCR3 to down-regulate the AKT-eNOS/NRF2 and up-regulate the ERK signaling pathways in HAECs. It is suggested that CCL7 plays a critical role in T2DM patients with vascular endothelial cell damages, and a promising therapeutic approach by targeting CCL7 is provided in the present disclosure.

### In Vivo Study

### Animal Models of DM

Six-week-old male BKS.Cg-m+/+Leprdb/JNarl mice were purchased from the National Laboratory Animal Center (Taipei, Taiwan). Mice were all acclimated for 2 weeks before experiments. For the treatment of hindlimb ischemia surgery, mice were injected intraperitoneally with CCL7 neutralizing antibody (0.1 or 1 μg; R&D, AF-456, Minneapolis, MN, USA) or IgG antibody (1 μg; R&D, AB-108, Minneapolis, MN, USA) three times per week for a month. Animals were raised according to the regulations of the Animal Care Committee of National Yang Ming Chiao Tung University (Taipei, Taiwan). The animal study was approved by the Animal Care Committee of National Yang Ming Chiao Tung University (IACUC No. 1100419).

### Hindlimb Ischemia Model

All mice were anesthetized via inhaled isoflurane. Mice were then shaved, and the surgical site was cleaned with 70% ethanol. The femoral artery and vein were separated from the femoral nerve, and femoral artery and vein were then ligated proximally and distally. The skin was closed with a noncontinuous suture. Hindlimb ischemia blood flow was analyzed by laser Doppler perfusion imaging (Moor Instruments Limited, Devon, UK) at days 0, 7, 14, 21, and 28 after surgery. The rate of reperfusion in the hind limb was calculated as a ratio of blood flow in the ischemic limb compared with the nonischemic limb for each mouse. The mice were sacrificed in deeply anesthesia induced via inhaled isoflurane. The gastrocnemius muscle from each leg was harvested for immunohistochemistry or protein expression analyses.

### Aortic Ring Assay

After sacrifice, the thoracic aortas were removed. The tissue was trimmed and the blood in the lumen was rinsed with saline. Aortic rings were then cut to 0.5 mm and embedded in 1 mg/mL type 1 rat tail collagen matrix (Millipore, 08115, Darmstadt, Germany) with incubation for 1 hour at 37°° C. Aortic rings were cultured using EBM-2 (Lonza, CC-3156, Basel, Switzerland) with supplements containing 2.5% bovine serum (Gibco, Carlsbad, CA, USA), 50 U/mL penicillin, 0.5 mg/mL streptomycin (Sigma-Aldrich, P4333, Darmstadt, Germany), and 30 ng/mL VEGF (Peprotech, 100-20, Rocky Hill, CT, USA) in 24-well plates for 7 days. After culture, aorta rings were photographed with a microscope (100×), and the branch area of aorta rings were calculated by image J software.

### Flow Cytometry

The mononuclear cells were suspended in saline and incubated with fluorescein isothiocyanate (FITC) anti-mouse Sca-1 (Invitrogen, 14-5981-82, Carlsbad, CA, USA) and phycoerythrin (PE) anti-mouse Flk-1 (VEGFR-2, Invitrogen,12-5821-82, Carlsbad, CA, USA) at room temperature for 30 minutes. A BD FACScalibur flow cytometer (BD, East Rutherford, NJ) was used, and data were analyzed with FloJo (Treestar). Data are presented as % gated, relative to control group.

### Measurement of Capillary Density in the Ischemic Limb

Histological analyses of capillary densities in the ischemic limb muscles were determined. Antigen retrieval was performed using 0.05 M sodium citrate buffer. Slides were then incubated at 4° C. overnight with the primary antibody to detect CD31 (Abcam, 124432, Waltham, MA, USA). The sample was washed with PBS solution and incubated with a secondary antibody (rabbit) for 2 hours at room temperature. The CD31-positive sites are shown in dark brown. Three cross-sections were analyzed for each animal; ten different fields from each tissue preparation were randomly selected, and visible capillaries were counted.

### Statistical Analysis

Results are expressed as the mean±standard deviation (SD). Data sets were analyzed with the unpaired Student's t test, followed by a Scheffe's multiple-comparison post hoc test. Statistical significance was set at p-values <0.05.

### Example 6: Treatment With CCL7 Neutralizing Antibody in db/db Type 2 Diabetic Mice

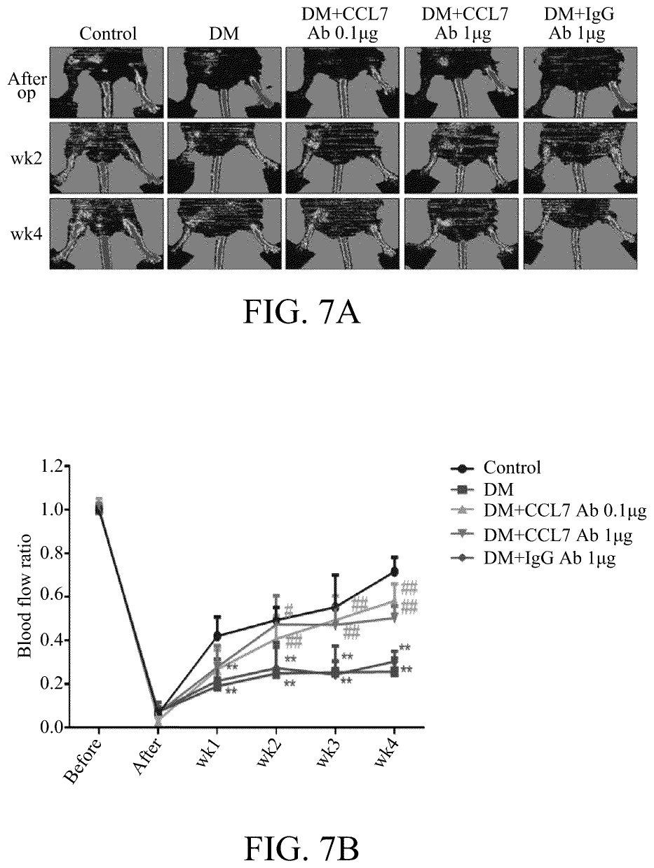

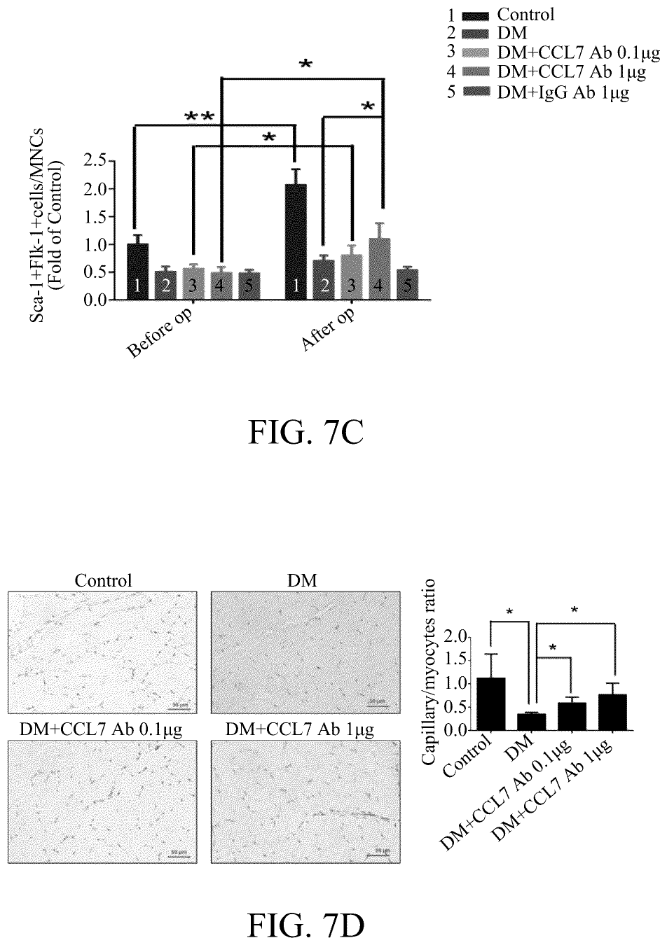

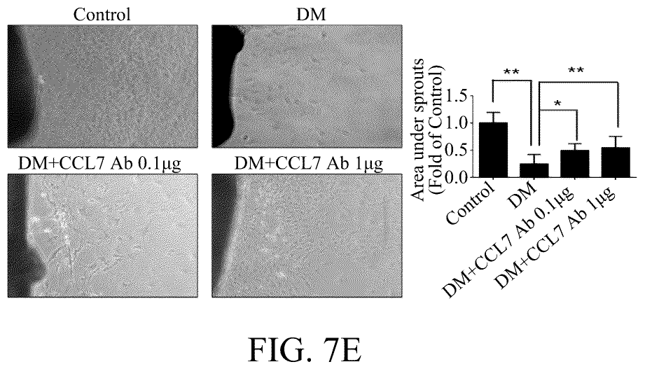

Blood flow in the ischemic hindlimb was equally reduced after hindlimb ischemia surgery in each group of mice. Perfusion recovery was attenuated in the type 2 DM mice compared with that in the non-DM control mice during the postoperative weeks. The blood flow ratio was significantly improved in both the 0.1 and 1 μg CCL7 antibody-treated mice compared with that in the untreated type 2 DM mice (FIG. 7A, 7B). The increased number of Sca-1+/Flk-1+EPC-like cells was attenuated in the untreated type 2 DM mouse group compared with that in the non-DM control mice at 2 days after ischemic surgery. The attenuation of the EPC-like cell number was recovered in the CCL7 antibody-treated group (FIG. 7C). Immunohistochemical analysis revealed that capillary densities in the ischemic limb were reduced in type 2 DM mice compared with those in non-DM control mice, and these values were increased in the CCL7 antibody-treated mice compared with those in the untreated type 2 DM mice (FIG. 7D). Additionally, angiogenesis in aortic ring cultures from CCL7 antibody-treated mice showed increased number of vessels sprouting compared with those in the untreated type 2 DM mice (FIG. 7E).

Foot blood flow monitored by laser Doppler imaging system in each group of mice. Please refer to FIG. 7A showing representative evaluation of the ischemic (right) and non-ischemic (left) hindlimbs, before, immediately after, and 4 weeks after the surgery. In FIG. 7A, red indicates normal perfusion and blue indicates a marked reduction in blood flow in the ischemic hindlimb. Circulating EPCs were measured by flow cytometry shown in FIG. 7C. As can be seen in FIG. 7D, anti-CD31 immunostaining showed that CCL7 antibody treatments significantly increased the number of capillaries. In FIG. 7E, angiogenesis in aortic ring cultures from CCL7 antibody-treated mice showed increased number of vessels sprouting. Non-DM control group, n=6; DM, n=6; DM+CCL7 antibody 0.1 μg group, n=6; DM+CCL7 antibody 1 μg group, n=6; DM+IgG group, n=4. *p<0.05, **p<0.01 compared with the control group. #p<0.05, ##p<0.01 compared with the DM group.

From the above, the experiments indicate that the inhibition of CCL7 may improve the functions of EPCs and HAECs of a subject. Also, the inhibition of CCL7 rescues the functions of EPCs or HAECs that are impaired due to high glucose stimulation. As improving the cell functions, the expressions of angiogenesis factors, such as p-eNOS, p-AKT, VEGF and SDF-1, are increased, thereby enhancing angiogenesis in the subject. Therefore, the method of using a CCL7 antagonist of the present discourse is useful in accelerating the angiogenic process, and thus effective in treating subjects suffering from PAD and diabetes.

Even though numerous characteristics and advantages of the present disclosure have been set forth in the foregoing description, together with details of the structure and features of the invention, the invention is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for preventing or treating a peripheral arterial disease (PAD) in a subject in need thereof, comprising administering an effective amount of a chemokine C-C motif ligand 7 (CCL7) antagonist to the subject to inhibit CCL7 activity.

2. The method according to claim 1, wherein the CCL7 antagonist is selected from a group consisting of a CCL7 neutralizing antibody, a CCL7 RNA interference (RNAi) agent, a C-C chemokine receptor type 1 antagonist, a C-C chemokine receptor type 2 antagonist, a C-C chemokine receptor type 3 antagonist, a C-C chemokine receptor type 5 antagonist, and a combination thereof.

3. The method according to claim 1, wherein the subject suffers from a disease inducing occlusion or potentiate stenosis of peripheral arteries.

4. The method according to claim 1, wherein the peripheral arterial disease is induced by at least one selected from the group consisting of diabetes, hypertension, radiation, toxins, atherosclerosis, thrombo-embolism, traumatic injury, vascular spasm, autoimmune disease, scleroderma, and vasculitis.

5. The method according to claim 4, wherein the diabetes is type 2 diabetes mellitus (T2DM).

6. The method according to claim 5, wherein the subject suffers from T2DM with vascular endothelial cell damages.

7. The method according to claim 1, wherein the subject is a human or an animal.

8. The method according to claim 1, wherein the effective amount of the CCL7 antagonist is from about 0.01 μg/kg to about 100 mg/kg.

9. The method according to claim 8, wherein the effective amount of the CCL7 antagonist is from about 0.1 μg/kg to about 1 mg/kg.

10. The method according to claim 1, wherein the CCL7 antagonist is administered to the subject orally, intraperitoneally, intravenously, intradermally, intramuscularly, subcutaneously, intrapleurally, or transdermally.

11. The method according to claim 6, wherein the administering enhances angiogenesis in the subject.

12. The method according to claim 6, wherein the administering protects endothelial cell functions in the subject.

13. The method according to claim 6, wherein the administering improves a diabetic vascular disease.
